(19) United States
(12) Patent Application Publication
(10) Pub. No.: US 2007/0203247 A1
(43) Pub. Date: Aug. 30, 2007

(54) EPINEPHRINE DOSING REGIMENS

(76) Inventors: Elaine Phillips, San Diego, CA (US); Greg Malone, Encinitas, CA (US)

Correspondence Address: WILSON SONSINI GOODRICH & ROSATI, 650 PAGE MILL ROAD, PALO ALTO, CA, 94304-1050, US

(21) Appl. No.: 11/680,533

(22) Filed: Feb. 28, 2007

Related U.S. Application Data
(60) Provisional application No. 60/743,381, filed on Feb. 28, 2006.

(51) Int. Cl. A61K 31/137

(52) U.S. Cl. 514/649

(57) ABSTRACT

The present invention relates to methods for administering a first dose of epinephrine solution and an optional second dose of epinephrine solution. Also provided herein are kits useful in these methods.

### RELATED APPLICATIONS

This application claims the benefit of U.S. Provisional Application No. 60/743,381, filed Feb. 28, 2006, which is hereby incorporated by reference in its entirety.

### BACKGROUND OF THE INVENTION

Allergic emergencies, such as anaphylaxis, are a growing concern, given the increasing awareness of members of the public of their frequency and potential severity. Anaphylaxis is a sudden, severe, systemic allergic reaction that can be fatal, in many cases, if left untreated. Anaphylaxis can involve various areas of the body, such as the skin, respiratory tract, gastrointestinal tract, and cardiovascular system. Acute symptoms generally occur from within about a minute to about two hours after contact with the allergy-causing substance, but in rare instances onset may be delayed by as much as eight hours. Contact with anaphylaxis-inducing agents, and the severity of the resulting anaphylactic reaction, can be extremely unpredictable. Accordingly, allergists recommend that persons who have a personal or family history of anaphylaxis, or a risk of anaphylaxis, be prepared to self-administer emergency treatment at all times. Additionally, adults charged with caring for children who are at risk for anaphylaxis should also be prepared to administer anti-anaphylactic first aid.

The symptoms of anaphylaxis frequently include one or more of the following, which generally occur within about 1 to about 15 minutes of exposure to the antigen: agitation, a feeling of uneasiness, flushing, palpitations, paresthesias, pruritus, throbbing in the ears, coughing, sneezing, urticaria, angioedema, difficulty breathing due to laryngeal edema or bronchospasm, nausea, vomiting, abdominal pain, diarrhea, shock, convulsions, incontinence, unresponsiveness and death. An anaphylactic reaction may include cardiovascular collapse, even in the absence of respiratory symptoms.

According to the Merck Manual, immediate treatment with epinephrine is imperative for the successful treatment of anaphylaxis. Merck Manual, 17^{th} Ed., 1053-1054 (1999). The recommended dose of epinephrine for the treatment of anaphylaxis is about 0.01 mg/Kg: usually about 0.3 to 0.5 mL of a 1:1000 dilution of epinephrine in a suitable carrier. While the dose may be given manually, either subcutaneously or intramuscularly, in recent years automatic injectors have become an accepted first aid means of delivering epinephrine. It is recommended that persons at risk of anaphylaxis, and persons responsible for children at risk for anaphylaxis, maintain one or more automatic epinephrine injectors in a convenient place at all times. It is further recommended that, if the symptoms of anaphylaxis persist after the first dose of epinephrine is injected, the patient should be treated with a second dose of epinephrine (for adults, about 0.3 mL of the 1:1000 dilution).

Current dosing regimens do not take into consideration the varying sizes or conditions of individuals in the population. For example, the delivery of 0.15 mg to a very small infant may not be safe, including where a second dose of 0.15 mg is delivered when the child does not respond to the first dose. Additionally, where the child is very small, the delivery of 0.15 mL (the amount of solution necessary when the concentration of the solution is 1 mg/ml epinephrine) may cause severe discomfort, which can lead to poor patient compliance or non-compliance. Similarly, the delivery of a 0.5 mg dose may not be sufficient to treat individuals larger than the average adult size. Moreover, many adults may be better treated by administering dosing regimens which require either the first dose, the second dose, or both doses of epinephrine to have a volume of greater than about 0.5 mL. Further, patients in at-risk populations, such as elderly patients or patients suspected or known to be at risk of heart attack, may require dosing regimens comprising higher or lower doses of epinephrine than are currently available. Thus, there remains a need for a method of treating anaphylaxis in persons in at-risk populations, persons above the average adult size, as well as methods for treating anaphylaxis in children of all ages and sizes. There is also a need for epinephrine dosing regimens incorporating a single device capable of delivering two such smaller or larger doses to these patients.

### SUMMARY OF THE INVENTION

The present invention meets the foregoing and related needs by providing an improved method of treating allergic emergencies, such as anaphylaxis, with epinephrine in patients where current treatments are not ideal.

In certain aspects of the present invention, provided herein are methods of treating an allergic emergency in a patient, e.g., a small child, comprising the step of injecting into a patient in need there of a first dose of an epinephrine solution comprising less than about 0.15 mg and optionally subsequently injecting into the patient a second dose of an epinephrine solution, wherein the first and second doses are administered from a single device. In alternate embodiments, the first and second doses described herein are administered from separate devices. In certain embodiments, the amount of epinephrine in the first dose is about the same as the amount of epinephrine in the second dose. In some embodiments, the amount of epinephrine in the first dose is about 0.1 mg or about 0.05 mg. In some embodiments, the amount of epinephrine in the second dose is about 0.1 mg or about 0.05 mg.

In certain other embodiments of the methods described herein, the amount of epinephrine in the first dose is greater than the amount of epinephrine in the second dose. In some embodiments, the amount of epinephrine in the second dose is about 0.1 mg or about 0.05 mg.

In still other embodiments of the methods described herein, the amount of epinephrine in the first dose is less than the amount of epinephrine in the second dose. In some embodiments, the amount of epinephrine in the second dose is at least about 0.15 mg or at least about 0.3 mg.

In yet other embodiments, the concentration of epinephrine in the first dose and/or second dose is about 2.0 mg of epinephrine per mL, or about 1.5 mg of epinephrine per mL, or about 1.0 mg of epinephrine per mL, or about 0.5 mg of epinephrine per mL, or about 0.33 mg of epinephrine per mL, or about 0.25 mg of epinephrine per mL, or about 0.2 mg of epinephrine per mL. In varying embodiments, the volume of the first dose is different from the volume of the second dose administered and/or the concentration of epinephrine in the first dose is different from the concentration of the second dose administered. Additionally, in some embodiments three or more doses of epinephrine solution are administered.

Within this aspect of the invention, the present methods are also directed to the treatment of a small child, wherein the weight of the small child can be, for example, less than about 30 kg, less than about 15 Kg, less than about 12 Kg, less than about 10 Kg, less than about 8 Kg, or less than about 5 Kg. Alternatively, the small child can, for example, have a weight of between about 4 to about 8 Kg, or between about 5 to about 10 Kg, or between about 10 to about 15 Kg, or between about 5 to about 15 Kg. In some embodiments, the child is greater than about 15 Kg in weight or between about 15-17 Kg, 15-20 Kg, 20-25 Kg, or 25-30 Kg, and the method comprises administering a first dose of an epinephrine solution comprising less than about 0.15 mg of epinephrine followed by administering a second dose of an epinephrine solution, wherein the first and second doses are administered from a single device. In alternate embodiments, the first and second doses described herein are administered from separate devices.

In other aspects of the present invention, provided herein are methods of treating an allergic emergency in a patient, e.g., an adult, comprising the step of injecting into patient in need there of a first dose of an epinephrine solution comprising at least about 0.5 mg epinephrine and optionally subsequently injecting into the patient a second dose of an epinephrine solution, wherein the first and second doses are administered from a single device. In alternate embodiments, the first and second doses described herein are administered from separate devices. In certain embodiments, the amount of epinephrine in the first dose is about the same as the amount of epinephrine in the second dose. In some embodiments, the amount of epinephrine in the first dose is about 0.5 mg, about 0.75 mg, about 1 mg, or about 0.5 mg to about 1.0 mg. In some embodiments, the amount of epinephrine in the second dose is about 0.5 mg, about 0.75 mg, about 1 mg, or about 0.5 mg to about 1.0 mg.

In certain other embodiments of the methods described herein, the amount of epinephrine in the first dose is greater than the amount of epinephrine in the second dose. In some embodiments, the amount of epinephrine in the second dose is about 0.5 mg, about 0.3, about 0.15 mg, or less than 0.15 mg.

In still other embodiments of the methods described herein, the amount of epinephrine in the first dose is less than the amount of epinephrine in the second dose. In some embodiments, the amount of epinephrine in the second dose is at least about 0.75 mg or at least about 1.0 mg.

In yet other embodiments, the concentration of epinephrine in the first dose and/or second dose is about 2 mg of epinephrine per mL, or about 1.5 mg of epinephrine per mL, or about 1 mg of epinephrine per mL, or about 0.5 mg of epinephrine per mL, or about 0.33 mg of epinephrine per mL, or about 0.25 mg of epinephrine per mL, or about 0.2 mg of epinephrine per mL. In varying embodiments, the volume of the first dose is different from the volume of the second dose administered or the concentration of epinephrine in the first dose may be different from the concentration of the second dose administered.

Within this aspect of the invention, the present methods are also directed to the treatment of an adult, wherein the weight of the adult can be, for example, greater than about 50 Kg, greater than about 60 Kg, greater than about 70 Kg, greater than about 80 Kg, greater than about 90 Kg, greater than about 100 Kg, greater than about 110 Kg, greater than about 120 Kg, greater than about 130 Kg, greater than about 140 Kg, or greater than about 150 Kg. Alternatively, the adult can, for example, have a weight of between about 50 to about 200 Kg, or about 50 to about 180 Kg, or about 50 to about 150 Kg, or about 50 to about 100 Kg, or about 70 to about 150 Kg, or about 50 to about 60 Kg, or about 60 to about 70 Kg, or about 70 to about 80 Kg, or about 80 to about 90 Kg, or about 90 to about 100 Kg, or about 100 to about 110 Kg, or about 110 to about 120 Kg, or about 120 to about 130 Kg, or about 130 to about 140 Kg, or about 140 to about 150 Kg. In other embodiments, the weight of the adult is less than about 50 Kg, and the method comprises administering a first dose of an epinephrine solution comprising at least about 0.5 mg of epinephrine followed by administering a second dose of an epinephrine solution, wherein the first and second doses are administered from a single device. In still other embodiments, the methods comprise administering a first dose of an epinephrine solution comprising at least about 0.5 mg of epinephrine followed by administering a second dose of an epinephrine solution, wherein the first and second doses are administered from separate devices. In an alternative embodiment, the first dose of epinephrine solution comprises greater than about 0.5 mg epinephrine and the second dose of epinephrine solution comprises about 0.5 mg.

As described above, the methods provided herein comprise injecting into a patient a first dose of epinephrine and optionally subsequently injecting a second dose of epinephrine, wherein the first and second doses are administered from a single device. In certain embodiments, the first dose may be delivered by either an automatic injection or a manual injection, and the second dose may be delivered by either an automatic injection or a manual injection. For example, both the first dose and second dose can be delivered by automatic injection from a single device, both the first dose and the second dose can be delivered by manual injection from a single device, the first dose can be administered by automatic injection and the second dose can be delivered by manual injection from a single device, or the first dose can be administered by manual injection and the second dose by automatic injection from a single device. In alternate embodiments, the first and second doses described herein are administered from separate devices.

As described above, the methods provided herein comprise injecting into a patient a first dose of epinephrine and optionally subsequently injecting a second dose of epinephrine, wherein the first and second doses are administered from a single device. In certain embodiments, the first dose may be injected either subcutaneously or intramuscularly, and the second dose may be injected either subcutaneously or intramuscularly. For example, both the first dose and second dose can be injected subcutaneously from a single device, both the first dose and the second dose can be injected intramuscularly from a single device, the first dose can be injected subcutaneously and the second dose can be injected intramuscularly from a single device, or the first dose can be injected intramuscularly and the second dose can be injected subcutaneously from a single device. In alternate embodiments, the first and second doses described herein are administered from separate devices.

In other embodiments of the present invention, the methods provided herein comprise injecting into a patient a first dose of epinephrine and optionally subsequently injecting a second dose of epinephrine from a single device, wherein the second dose can be injected less than 30 minutes after the first dose. For example, the second dose can be injected less than 20 minutes after the first dose, or the second dose can be injected less than 10 minutes after the first dose. In still other embodiments, the second dose can be injected between about 2 to about 10 minutes after the first dose, or the second dose can be injected between about 2 to about 5 minutes after the first dose. In alternate embodiments, the first and second doses described herein are administered from separate devices.

In still other embodiments of the present invention, the methods provided herein comprise injecting into a patient a first dose of epinephrine and optionally subsequently injecting a second dose of epinephrine from a single device, wherein the first dose may be self-administered by the patient or administered by someone other than the patient, and the second dose may be self-administered by the patient or administered by someone other than the patient. For example, both the first dose and second dose can be self-administered by the patient from a single device, both the first dose and the second dose can be administered by someone other than the patient from a single device, the first dose can be self-administered by the patient and the second dose can be administered by someone other than the patient from a single device, or the first dose can be administered by someone other than the patient and the second dose can be self-administered by the patient from a single device. In alternate embodiments, the first and second doses described herein are administered from separate devices.

The invention further provides a kit or packaging system for treatment of allergic emergencies, such as anaphylaxis. In certain aspects, the kit or packaging system includes a first injectable dosage form comprising an epinephrine solution comprising less than about 0.15 mg of epinephrine and a second injectable dosage form comprising epinephrine. In certain other aspects, the kit or packaging system includes a first injectable dosage form comprising an epinephrine solution comprising at least about 0.5 mg of epinephrine and a second injectable dosage form comprising epinephrine. In certain embodiments of the kits or packaging systems described above, the kits or packaging systems are able to hold and/or store the first and second injectable dosage forms comprising epinephrine and further comprise written instructions for administering the first and second injectable dosage forms comprising epinephrine to treat anaphylaxis. In certain other embodiments, the first and second injectable dosage forms comprising epinephrine of the kit or packaging system can be administered from a single device. In still other embodiments, the first and second injectable dosage forms comprising epinephrine of the kit or packaging system can be administered from separate devices. The amounts of epinephrine contained in the kit are as described above, as well as throughout the specification.

### INCORPORATION BY REFERENCE

All publications and patent applications mentioned in this specification are herein incorporated by reference to the same extent as if each individual publication or patent application was specifically and individually indicated to be incorporated by reference.

### DETAILED DESCRIPTION OF THE INVENTION

A better understanding of the features and advantages of the present invention will be obtained by reference to the following detailed description that sets forth illustrative embodiments, in which the principles of the invention are utilized.

The present invention provides methods for treating allergic emergencies, such as anaphylaxis, comprising the step of injecting into a patient in need there of a first dose of an epinephrine solution comprising less than about 0.15 mg and optionally subsequently injecting into the patient a second dose of an epinephrine solution, wherein the first and second doses are administered from a single device. In alternate embodiments, the first and second doses described herein are administered from separate devices.

The present invention further provides methods for treating allergic emergencies, such as anaphylaxis, comprising the step of injecting into a patient in need there of a first dose of an epinephrine solution comprising at least about 0.5 mg and optionally subsequently injecting into the patient a second dose of an epinephrine solution, wherein the first and second doses are administered from a single device. In alternate embodiments, the first and second doses described herein are administered from separate devices.

Furthermore, the invention provides kits or packaging systems comprising the first and second injectable dosage forms comprising the amounts of epinephrine as set forth throughout the present specification useful for such methods.

As used herein, the term “about” is used synonymously with the term “approximately.” As one of ordinary skill in the art would understand, the exact boundary of “about” will depend on the component of the composition. Illustratively, the use of the term “about” indicates that values slightly outside the cited values, i.e., plus or minus 0.1% to 10%, are intended to be included within the cited values.

As used herein, the terms “comprising,” “including,” “such as,” and “for example” are used in their open, non-limiting sense.

As used herein, “anaphylaxis” means an acute and severe allergic reaction to an allergen (antigen).

“Treatment of anaphylaxis” means ameliorating or alleviating the symptoms of anaphylaxis. Such treatment may be, and in most cases is, temporary. For example, in embodiments of the invention the method, device or kit of the invention will provide emergency relief from the symptoms of anaphylaxis for a time sufficient for the patient to seek professional medical assistance.

“Treatment of an allergic emergency” includes treatment of anaphylaxis. In addition, treatment of allergic emergency includes treatment of other allergic conditions that may be treated with epinephrine. For example, the symptoms of anaphylactoid reactions to drugs closely mimic those of anaphylaxis and are treated in a similar manner. In cases where it is not clear whether the reaction is a systemic immunological response (anaphylaxis) or a systemic toxic response (anaphylactoid reaction), the accepted first line of treatment is with epinephrine. In this sense, treatment of an allergic emergency encompasses treatment of anaphylaxis, an anaphylactic response or both.

Epinephrine Solution Dosing Regimens

Dosing in Children or Small Adults

In certain aspects, the present invention provides a method of treating an allergic emergency in a patient, such as anaphylaxis, comprising administering to the patient a first injectable dose of an epinephrine solution and optionally subsequently administering a second injectable dose of an epinephrine solution, wherein the first and second doses are from the same device. In other embodiments, the present invention provides a method of treating an allergic emergency in a patient, such as anaphylaxis, comprising administering to the patient two injectable doses of an epinephrine solution from different devices.

In each of these embodiments, the methods include injecting a first dose of an epinephrine solution comprising less than about 0.15 mg of epinephrine and optionally subsequently injecting into the patient a second dose of epinephrine solution from the same device. In alternate embodiments, the first dose and optional second dose described herein are administered from separate devices. In some embodiments, the second dose of epinephrine solutions comprises about at least about 0.5 mg, or about 0.3 mg, or about 0.2 mg, or about 0.15 mg, or about 0.1 mg, or about 0.05 mg, or about 0.025 mg of epinephrine. In other embodiments, the second dose of epinephrine solution comprises between about 0.025 to 1.0 mg, or between about 0.05 to 0.75 mg, or between about 0.05 to 0.5 mg, or between about 0.05 to 0.3 mg, or between about 0.05 to 0.2 mg, or between about 0.1 to 0.2 mg, or between about 0.05 to about 0.1 mg, or between about 0.1 to about 0.15 mg epinephrine.

Also provided herein are methods comprising the steps of injecting a first dose of an epinephrine solution comprising about 0.1 mg of epinephrine and optionally subsequently injecting into the patient a second dose of an epinephrine solution from the same device. In alternate embodiments, the first dose and optional second dose described herein are administered from separate devices. In some embodiments, the second dose of an epinephrine solution comprises about at least about 0.5 mg, or about 0.3mg, or about 0.2 mg, or about 0.15 mg, or about 0.1 mg, or about 0.05 mg, or about 0.025 mg of epinephrine. In other embodiments, the second dose of epinephrine solution comprises between about 0.025 to 1.0 mg, or between about 0.05 to 0.75 mg, or between about 0.05 to 0.5 mg, or between about 0.05 to 0.3 mg, or between about 0.05 to 0.2 mg, or between about 0.1 to 0.2 mg, or between about 0.05 to about 0.1 mg, or between about 0.1 to about 0.15 mg epinephrine.

Provided herein are also methods comprising the steps of injecting a first dose of an epinephrine solution comprising between about 0.05 mg to about 0.2 mg of epinephrine and optionally subsequently injecting into the patient a second dose of an epinephrine solution from the same device. In alternate embodiments, the first dose and optional second dose described herein are administered from separate devices. In some embodiments, the second dose of an epinephrine solution comprises about at least about 0.5 mg, or about 0.3 mg, or about 0.2 mg, or about 0.15 mg, or about 0.1 mg, or about 0.05 mg, or about 0.025 mg of epinephrine. In other embodiments, the second dose of epinephrine solution comprises between about 0.025 to 1.0 mg, or between about 0.05 to 0.75 mg, or between about 0.05 to 0.5 mg, or between about 0.05 to 0.3 mg, or between about 0.05 to 0.2 mg, or between about 0.1 to 0.2 mg, or between about 0.05 to about 0.1 mg, or between about 0.1 to about 0.15 mg epinephrine. In still other embodiments, the first dose comprises between about 0.05 to about 0.15 mg, or between about 0.05 to about 0.1 mg epinephrine. In each of these embodiments, i.e., where the first dose is varied, the second dose can also be varied as described above.

In some embodiments, aside from epinephrine, the epinephrine solution also contains at least one pharmaceutically inactive ingredient, such as a preservative (e.g., sodium bisulfite), a pH buffer or buffer system, an agent for adjusting osmolality (such as to establish or maintain isotonicity with the tissue in which the solution is to be injected), or a mixture of two or more of the foregoing. Thus, as used herein, unless otherwise defined, the term “epinephrine solution” means an aqueous solution of epinephrine, which optionally comprises one or more additional ingredients other than epinephrine and water, such as a preservative, a buffer, and/or an agent for adjusting osmolality. As understood by one of skill in the art, different epinephrine solution concentrations can be used by adjusting the volume of epinephrine solution injected. For example, in some embodiments, the concentration of the epinephrine solution is about 0.2 mg of epinephrine per mL, or about 0.25 mg of epinephrine per mL of solution, or about 0.33 mg of epinephrine per mL, or about 0.5 mg of epinephrine per mL of solution, or about 0.75 mg of epinephrine per mL of solution, or about 1 mg of epinephrine per mL of solution, or about 1.25 mg of epinephrine per mL of solution, or about 1.5 mg of epinephrine per mL of solution, or about 1.75 mg of epinephrine per mL of solution, or about 2.0 mg of epinephrine per mL of solution. Contemplated by and included within the scope of the present invention is the administration of different epinephrine concentrations in different doses. Also contemplated by and included within the scope of the present invention is the administration of different volumes of epinephrine solution in different doses.

In some of these embodiments, the method includes automatically injecting both the first and the second doses with an injection device. In other embodiments, the method includes manually injecting both the first and the second doses with an injection device. In yet other embodiments, the method includes injecting one of the doses automatically, and one of the doses manually, with an injection device, for example, the first dose is delivered by automatic injection and the second dose is delivered by manual injection from the same device.

The first dose may be self-administered by the patient, or may be administered by someone other than a patient, such as a caretaker or a medical professional. Additionally, the second dose may be self-administered by the patient, or may be administered by someone other than a patient; such as a caretaker or medical professional. It is necessary that the patient monitor his or her symptoms, or that the person caring for the patient monitors the patient's symptoms directly. In cases where the symptoms of anaphylaxis are not suitably ameliorated by administration of the first injection of the epinephrine solution (whether by manual or automatic injection), it will be necessary to administer the second dose. In some embodiments, it may be necessary to administer a third dose, optionally a fourth dose, and optionally a fifth dose. Additionally, in cases where the patient is unable to obtain professional medical assistance before the beneficial effects of the first dose begin to subside, it may be necessary to administer a second dose (whether by manual or automatic injection). In some embodiments, a subsequent dose is administered less than about 60 minutes after the first dose, e.g. less than about 45 minutes, less than about 30 minutes, less than about 20 minutes, less than about 10 minutes, or less than about 5 minutes after the first dose. In alternative embodiments, the second dose is administered within about 2-10 minutes, or about 2-5 minutes, about 5-10 minutes or about 2-15 minutes of the previous dose. For example, the second dose can administered less than about 60 minutes after the first dose, e.g., less than about 45 minutes, less than about 30 minutes, less than about 20 minutes, less than about 10 minutes, or less than about 5 minutes after the first dose. In alternative embodiments, the second dose is administered within about 2-10 minutes, or about 2-5 minutes or about 5-10 minutes or about 2-15 minutes of the first dose.

The smaller doses of epinephrine solution described above, i.e., where the first and second doses are as described above, but in any event, where at least one of the first or second doses comprises less than about 0.15 mg of epinephrine, are especially suitable for treating smaller patients, who may find larger injections uncomfortable, painful, intimidating or even harmful where the individual, such as a child, is young or very small. Thus, in some of the embodiments described herein, the patient receiving the treatment weighs less than about 30 Kg. In particular embodiments, the patient weighs less than about 20 Kg, or less than about 15 Kg, or less than about 10 Kg, or less than about 5 Kg. In other embodiments, the patient is a child less than about 12 years old, or less than about 9 years old, or less than about 7 years old, or less than about 5 years old, or less than about 3 years old, or less than about 1 year old, or about 1 to 3 years old, or about 1 to 5 years old, or about 1 to 12 years old, or about 2 to 5 years old, or about 2 to 12 years old, or about 5 to 12 years old.

Dosing in Adults

In certain other aspects, the present invention provides a method of treating an allergic emergency, such as anaphylaxis, in a patient, comprising administering to the patient a first injectable dose of an epinephrine solution and optionally subsequently administering a second dose of an epinephrine solution, wherein the first and second doses are from the same device. In other embodiments, the present invention provides a method of treating an allergic emergency, such as anaphylaxis, in a patient, comprising administering to the patient two injectable doses of an epinephrine solution, wherein the first and second doses are from different devices.

In each of these embodiments, the methods include injecting a first dose of an epinephrine solution comprising at least about 0.5 mg of epinephrine and optionally subsequently injecting into the patient a second dose of an epinephrine solution from the same device. In alternate embodiments, the first dose and optional second dose described herein are administered from separate devices. In some embodiments, the second dose of an epinephrine solution comprises less than about 0.15 mg, or about 0.15 mg, or about 0.3 mg, or about 0.5 mg, or about 0.7 mg, or about 0.9 mg, or about 1.0 mg, or about 1.2 mg, or about 1.5 mg of epinephrine. In other embodiments, the second dose of epinephrine solution comprises between about 0.15 to 2.0 mg, or between about 0.3 mg to 2.0 mg, or between about 0.5 to 2.0 mg, or between about 0.7 to about 1.5 mg, or between about 0.5 to about 1.0 mg, or between about 0.7 to about 1.0 mg of epinephrine.

Also provided herein are methods comprising the steps of injecting a first dose of an epinephrine solution comprising at least about 0.7 mg of epinephrine and optionally subsequently injecting into the patient a second dose of an epinephrine solution from the same device. In alternate embodiments, the first dose and optional second dose described herein are administered from separate devices. In some embodiments, the second dose of an epinephrine solution comprises less than about 0.15 mg, or about 0.15 mg, or about 0.3 mg, or about 0.5 mg, or about 0.7 mg, or about 0.9 mg, or about 1.0 mg, or about 1.2 mg, or about 1.5 mg of epinephrine. In other embodiments, the second dose of epinephrine solution comprises between about 0.15 to 2.0 mg, or between about 0.3 mg to 2.0 mg, or between about 0.5 to 2.0 mg, or between about 0.7 to about 1.5 mg, or between about 0.5 to about 1.0 mg, or between about 0.7 to about 1.0 mg of epinephrine.

Provided herein are methods comprising the steps of injecting a first dose of an epinephrine solution comprising at least about 0.5 mg of epinephrine and optionally subsequently injecting into the patient a second dose of an epinephrine solution comprising at least about 0.5 mg of epinephrine from the same device. In alternate embodiments, the first dose and optional second dose described herein are administered from separate devices.

In some embodiments, the first dose of an epinephrine solution comprises about 0.5 mg, or about 0.7 mg, or about 0.9 mg, or about 1.0 mg, or about 1.2 mg, or about 1.5 mg of epinephrine and the second dose of epinephrine solution comprises about 0.5 mg, or about 0.7 mg, or about 0.9 mg, or about 1.0 mg, or about 1.2 mg, or about 1.5 mg of epinephrine. In additional embodiments, the first dose of an epinephrine solution comprises between about 0.5 to 2.0 mg, or between about 0.7 to about 1.5 mg, or between about 0.5 to about 1.0 mg, or between about 0.7 to about 1.0 mg of epinephrine and the second dose of epinephrine solution comprises between about 0.5 to 2.0 mg, or between about 0.7 to about 1.5 mg, or between about 0.5 to about 1.0 mg, or between about 0.7 to about 1.0 mg of epinephrine.

Also provided herein are methods comprising the steps of injecting a first dose of an epinephrine solution comprising between about 0.5 mg to about 2.0 mg of epinephrine and optionally subsequently injecting into the patient a second dose of an epinephrine solution from the same device. In alternate embodiments, the first dose and optional second dose described herein are administered from separate devices. In some embodiments, the second dose of an epinephrine solution comprises about less than about 0.15 mg, or about 0.15 mg, or 0.3 mg, or about 0.5 mg, or about 0.7 mg, or about 0.9 mg, or about 1.0 mg, or about 1.2 mg, or about 1.5 mg of epinephrine. In other embodiments, the second dose of an epinephrine solution comprises between about 0.3 to 2.0 mg, or between about 0.7 to about 1.5 mg, or between about 0.5 to about 1.0 mg, or between about 0.7 to about 1.0 mg of epinephrine. In still other embodiments, the first dose comprises between about 0.15 to 2.0 mg, or between about 0.3 to 2.0 mg, or between about 0.7 to about 1.5 mg, or between about 0.5 to about 1.0 mg, or between about 0.7 to about 1.0 mg of epinephrine. In each of these embodiments, i.e., where the first dose is varied, the second dose can be varied as described above.

In some embodiments, aside from epinephrine, the epinephrine solution also contains at least one pharmaceutically inactive ingredient, such as a preservative (e.g., sodium bisulfite), a pH buffer or buffer system, an agent for adjusting osmolality (such as to establish or maintain isotonicity with the tissue in which the solution is to be injected), or a mixture of two or more of the foregoing. Thus, as used herein, unless otherwise defined, the term “epinephrine solution” means an aqueous solution of epinephrine, which optionally comprises one or more additional ingredients other than epinephrine and water, such as a preservative, a buffer, and/or an agent for adjusting osmolality. As understood by one of skill in the art, different epinephrine solution concentrations can be used by adjusting the volume of epinephrine solution injected. For example, in some embodiments, the concentration of the epinephrine solution is about 0.2 mg of epinephrine per mL of solution, or about 0.25 mg of epinephrine per mL of solution, or about 0.33 mg of epinephrine per mL of solution, or about 0.5 mg of epinephrine per mL of solution, or about 0.75 mg of epinephrine per mL of solution, or about 1 mg of epinephrine per mL of solution, or about 1.25 mg of epinephrine per mL of solution, or about 1.5 mg of epinephrine per mL of solution, or about 1.75 mg of epinephrine per mL of solution, or about 2.0 mg of epinephrine per mL of solution. Contemplated by the present invention is the administration of different epinephrine concentrations in different doses. Also contemplated by the present invention is the administration of different volumes of epinephrine solution in different doses.

In some of these embodiments, the method includes automatically injecting both the first and the second doses with an injection device. In other embodiments, the method includes manually injecting both the first and the second doses with an injection device. In yet other embodiments, the method includes injecting one of the doses automatically, and one of the doses manually, with an injection device, for example, the first dose is delivered by automatic injection and the second dose is delivered by manual injection from the same device.

The first dose may be self-administered by the patient, or may be administered by someone other than a patient, such as a caretaker or a medical professional. Additionally, the second dose may be self-administered by the patient, or may be administered by someone other than a patient, such as a caretaker or medical professional. It is necessary that the patient monitor his or her symptoms, or that the person caring for the patient monitors the patient's symptoms directly. In cases where the symptoms of anaphylaxis are not suitably ameliorated by administration of the first injection of the epinephrine solution (whether by manual or automatic injection), it will be necessary to administer the second dose. Additionally, in cases where the patient is unable to obtain professional medical assistance before the beneficial effects of the first dose begin to subside, it will be necessary to administer a second dose (whether by manual or automatic injection). In other embodiments, a subsequent dose is administered less than about 60 minutes after the first dose, e.g., less than about 45 minutes, less than about 30 minutes, less than about 20 minutes, less than about 10 minutes, or less than about 5 minutes after the first dose. In alternative embodiments, the second dose is administered within about 2-10 minutes, or about 2-5 minutes, about 5-10 minutes, or about 2-15 minutes of the previous dose. For example, the second dose can administered less than about 60 minutes after the first dose, e.g. less than about 45 minutes, less than about 30 minutes, less than about 20 minutes, less than about 10 minutes, or less than about 5 minutes after the first dose. In alternative embodiments, the second dose is administered within about 2-10 minutes, about 2-5 minutes, or about 5-10 minutes, or about 2-15 minutes of the first dose.

The larger doses of epinephrine solution described above, i.e., where the first and second doses are as described above, but in any event at least one of the first or second doses comprises greater than about 0.5 mg epinephrine or both the first and second doses comprise about 0.5 mg of epinephrine, are especially suitable for treating larger patients, for whom smaller injections may not be suitable or provide sufficient relief from the symptoms of anaphylaxis. Thus, in some of the embodiments described herein, the patient receiving the treatment weighs more than about 50 Kg. In particular embodiments, the patient weighs more than about 60 Kg, or more than about 70 Kg, or more than about 80 Kg, or more than about 90 Kg, or more than about 100 Kg, or more than about 110 Kg, or more than about 120 Kg, or more than about 130 Kg, or more than about 140 Kg, or more than about 150 Kg. In other embodiments, the patient weight between about 50 to about 200 Kg, or between about 50 to about 100 Kg, or between about 100 to about 150 Kg, or between about 150 to about 200 Kg, or between about 50 to about 60 Kg, or between about 60 to about 70 Kg, or between about 70 to about 80 Kg, or between about 80 to about 90 Kg, or between about 90 to about 100 Kg, or between about 100 to about 110 Kg, or between about 110-120 Kg, or between about 120-130 Kg, or between about 130-140 Kg, or between about 140-150 Kg.

Multiple Dosing of Epinephrine Solution with Automatic-Automatic Devices

As described throughout the specification, the first and second doses described in the methods provided herein can each be automatically injected from the same device. Devices useful for these embodiments are those having the capability of injecting a plurality of medicament dosages. Examples of automatic-automatic injectors useful in the methods described herein can be found throughout the art and include, for example, those described in U.S. Pat. Nos. 3,572,336; 4,226,235; 4,031,893; 4,394,863; 4,723,937; 5,358,489; 5,665,071; 5,540,664; 3,882,863; 3,721,301; and 4,226,235 (each of which is incorporated by reference herein in their entirety).

Additionally, devices having a single chamber for the epinephrine solution used in both the first and second doses, a single spring but capable of administering two different doses of medication, and a mechanism to prevent the administration of both the first and second dose at the same time are also useful in the present invention. In these embodiments, preferably the needle of the device retracts back when it is removed from the patient after automatic administration of the first dose. Alternatively, the needle of the device can be removed after automatic administration of the first dose. The tension left in the spring of such devices is sufficient to re-inject the same needle or a new needle into the patient upon administration of the second dose.

Multiple Dosing of Epinephrine Solution with Automatic-Manual Devices

As described throughout the specification, a device useful in the methods described herein is one that is capable of delivering the first dose of epinephrine solution automatically and delivering the second dose of epinephrine solution manually from the same device. Devices useful for these embodiments are those having the capability of injecting a plurality of medicament dosages. Examples of automatic-manual injectors useful in the methods described herein can be found throughout the art and include, for example, those described in U.S. Pat. Nos. 5,695,472; 5,358,489; 5,540,664; 5,899,669; and 5,665,071 (each of which is incorporated by reference herein in their entirety).

Multiple Dosing of Epinephrine Solution with Manual-Automatic Devices

As described throughout the specification, a device useful in the methods described herein is one that is capable of delivering the first dose of epinephrine solution manually and delivering the second dose of epinephrine solution automatically from the same device. Devices useful for these embodiments are those having the capability of injecting a plurality of medicament dosages. Examples of manual-automatic injectors useful in the methods described herein can be found throughout the art. Moreover, one of skill in the art would, with the teachings found throughout this application and those in the art, understand how to make and use a manual-automatic device useful in the present invention.

The following is a description of but one first dose manual injection, and a second dose automatic injection device useful in the present invention. The described uses are both possible using the same or similar procedures with a single fixed needle syringe or a double needle syringe as those devices are known and described in the art. To perform the first dose manual injection, the user (patient or someone other than the patient) manually inserts the forward needle into the flesh of the patient and depresses the plunger rod, preferably with the thumb. For the first dose manual injection, a stop collar is employed to stop the plunger assembly of the syringe subassembly for injecting the desired amount of drug. After injection, the stop collar is removed from the syringe subassembly to allow the plunger's further movement for a second dose automatic injection.

Multiple Dosing of Epinephrine Solution with Manual-Manual Devices

As described throughout the specification, the first and second doses described in the methods provided herein can each be manually injected from the same device. Devices useful for these embodiments are those having the capability of injecting a plurality of medicament dosages. Examples of manual-manual injectors useful in the methods described herein can be found throughout the art and include, for example, those described in U.S. Pat. Nos. 5,358,489; 5,540,664; 5,722,956; 5,232,459; and 5,665,071 (each of which is incorporated by reference herein in their entirety); PCT Application No. WO88/07874 (which is incorporated by reference herein in its entirety), and US Published Application No. 2003/0004467 (which is incorporated by reference herein in its entirety).

Multiple Dosing of Epinephrine Solution

As described throughout the specification and claims, the epinephrine solutions for use in the methods provided herein can be administered from the same or different devices. Specifically, the first dose can be administered from the same device as a subsequent dose (e.g., optional second, optional third, optional fourth, optional fifth, etc.). Alternatively, the first dose can be administered from a different device than a subsequent dose (e.g., optional second, optional third, optional fourth, optional fifth, etc.).

In these embodiments, each dose of an epinephrine solution contains an amount of epinephrine independently selected from about 0.025 mg, 0.05 mg, 0.1 mg, 0.15 mg, 0.2 mg, 0.25 mg, 0.3 mg, 0.4 mg, 0.45 mg, 0.5 mg, 0.6 mg, 0.7 mg, 0.8 mg, 0.9 mg, 1.0 mg, 1.1 mg, 1.2 mg, 1.3 mg, 1.4 mg, 1.5 mg, 1.6 mg, 1.7 mg, 1.8 mg, 1.9 mg, 2.0 mg, 2.1 mg, 2.2 mg, 2.3 mg, 2.4 mg, 2.5 mg. In alternative embodiments, each dose of epinephrine solution contains an amount of epinephrine independently selected from about 0.025-2.5 mg, or 0.025-2.0 mg, or 0.05-2.5 mg, or 0.05-0.2 mg, or 0.5-2.5 mg, or 0.5-1.0 mg, or 0.05-0.1 mg, or 0.1-0.2 mg, or 0.2-0.3 mg, or 0.3-0.4 mg, or 0.4-0.5 mg, or 0.6-0.7 mg, or 0.7-0.8 mg, or 0.8-0.9 mg, or 0.9-1.0 mg, or 1.0-1.1 mg, or 1.1-1.2 mg, or 1.2-1.3 mg, or 1.3-1.4 mg, or 1.4-1.5 mg, or 1.5-1.6 mg, or 1.6-1.7 mg, or 1.7-1.8 mg, or 1.8-1.9 mg, or 1.9-2.0 mg.

In some embodiments, the epinephrine doses increase in strength (e.g., the second dose contains more epinephrine than the first, and the third dose (when present) contains more epinephrine than the second dose). In other embodiments, the epinephrine doses decrease in strength (e.g., the second dose contains less epinephrine than the first, and the third dose (when present) contains less epinephrine than the second dose). In still other embodiments, each of the doses administered are about the same strength. In yet other embodiments, the epinephrine doses increase in strength and then decrease in strength (e.g., the second dose contains more epinephrine than the first dose and the third dose contains less epinephrine than the second dose)—the alternative is also possible where the epinephrine doses decrease in strength and then increase in strength (e.g., the second dose contains less epinephrine than the first dose and the third dose contains more epinephrine than the second dose).

In some embodiments, aside from epinephrine, the epinephrine solution also contains at least one pharmaceutically inactive ingredient, such as a preservative (e.g., sodium bisulfite), a pH buffer or buffer system, an agent for adjusting osmolality (such as to establish or maintain isotonicity with the tissue in which the solution is to be injected), or a mixture of two or more of the foregoing. Thus, as used herein, unless otherwise defined, the term “epinephrine solution” means an aqueous solution of epinephrine, which optionally comprises one or more additional ingredients other than epinephrine and water, such as a preservative, a buffer, and/or an agent for adjusting osmolality. As understood by one of skill in the art, different epinephrine solution concentrations can be used by adjusting the volume of epinephrine solution injected. For example, in some embodiments, the concentration of the epinephrine solution is about 0.2 mg of epinephrine per mL, or about 0.25 mg of epinephrine per mL of solution, or about 0.33 mg of epinephrine per mL, or about 0.5 mg of epinephrine per mL of solution, or about 0.75 mg of epinephrine per mL of solution, or about 1 mg of epinephrine per mL of solution, or about 1.25 mg of epinephrine per mL of solution, or about 1.5 mg of epinephrine per mL of solution, or about 1.75 mg of epinephrine per mL of solution, or about 2.0 mg of epinephrine per mL of solution. Contemplated by the present invention is the administration of different epinephrine concentrations in different doses. Also contemplated by the present invention is the administration of different volumes of epinephrine solution in different doses.

In embodiments where the administration of more than two doses is contemplated, the devices useful in the invention are those capable of administering more than two doses, e.g., three doses, four doses, five doses, etc. These devices include any combination of auto and manual devices, for example (but not limited to) auto-auto-auto, manual-manual-manual, or auto-auto-manual. In some embodiments, the different doses are administered from different devices (such as those described in the above sections entitled Multiple Dosing of Epinephrine Solutions with Automatic-Automatic Devices, Multiple Dosing of Epinephrine Solution with Automatic-Manual Devices, Multiple Dosing of Epinephrine Solution with Manual-Automatic Devices, and Multiple Dosing of Epinephrine Solution with Manual-Manual Devices). For example, where the method comprises administering three doses, two doses may be administered from the same device and one dose from a different device. Alternatively, all three doses may be administered from the same device, or all three doses may be administered from different devices. Some devices useful in the administration of two or more doses of epinephrine solution use multiple stop collars. For example, where three doses will be administered from the same device, that device may have two stop collars to allow for accurate administration of the three different doses, or even three stop collars, where the first stop collar is removed before a first dose of epinephrine solution is injected.

Additional Methods of Treatment

In addition to being useful for the treatment of children and adults as described above, the methods described herein are useful for treating specific patient classes, such as those patients in at-risk populations or those patients (including average-sized adults) for whom a physician or other healthcare provider determines that the methods, devices, and dosing regimens described herein are appropriate.

In certain aspects, the methods described herein are useful for treating allergic emergencies in patients who have heart disease, high blood pressure, or take medicines to treat heart conditions, where current epinephrine dosing regimens (e.g. 0.15 mg/dose or 0.3 mg/dose) may not be appropriate due to the patients' potential for increased sensitivity to epinephrine. Likewise, the methods described herein are also useful for treating patients with diabetes. Moreover, the methods described herein are useful for treating patient with thyroid problems. For each of these patient classes, the methods described in the above section entitled Dosing in Children or Small Adults, can be used.

The methods described herein are also useful for treating patients who are at risk for heart disease, high blood pressure, or diabetes such as the elderly, where current epinephrine dosing regimens (e.g. 0.15 mg/dose or 0.3 mg/dose) may not be appropriate due to the patients' potential for increased sensitivity to epinephrine. For each of these patient classes, the methods described in the above section entitled Dosing in Children or Small Adults, can be used.

The methods described herein are useful for treating patients currently taking one or more medications that may sensitize the patient to one or more side effects of epinephrine, where current epinephrine dosing regimens (e.g. 0.15 mg/dose or 0.3 mg/dose) may not be appropriate due to the patients' potential for increased sensitivity to epinephrine. For example, a patient taking a drug known to sensitize the heart to arrhythmias, may be administered the drug according to the methods described in the above section entitled Dosing in Children or Small Adults.

The methods describe herein are also useful for treating patients who have previously experienced one or more side effects associated with epinephrine, such as (very) high blood pressure, severe headache, blurred vision, flushed skin, increases difficult in breathing, fast or irregular heart beats, sweating, nausea and vomiting, pale skin, dizziness, weakness or muscle tremors, apprehension, nervousness, and anxiety, where current epinephrine dosing regimens (e.g. 0.15 mg/dose or 0.3 mg/dose) may not be appropriate due to the patients' potential for increased sensitivity to epinephrine. In these patients, the methods described in the above section entitled Dosing in Children or Small Adults, can be used.

Kits

The invention includes a kit or packaging system for administration of epinephrine to a patient in need thereof, such as a patient experiencing anaphylaxis, an anaphylactoid reaction or a set of symptoms resembling anaphylaxis or anaphylactoid reaction of unknown etiology but suspected of being an allergic emergency. The kit or packaging system includes an injector according to the present invention as well as such additional matter as may be necessary to ease administration of the epinephrine to the patient. In some embodiments of the invention, included in the kit or packaging system is an injector that provides a first dose and a second dose delivered by automatic injection from the same device. In other embodiments, included in the kit or packaging system is an injector that provides a first dose and a second dose delivered by manual injection from the same device, and in other embodiments, included in the kit or packaging system is an injector that provides one dose is administered by manual injection and the other dose by automatic injection, and in particular, the injector provides a first dose delivered by manual injection and a second dose delivered by automatic injection from the same device. In other embodiments of the invention, included in the kit or packaging system are two injectors as separate devices, wherein one injector provides a first dose and second injector provides a second dose, both of which are delivered by automatic injection. In other embodiments, included in the kit or packaging system are two injectors as separate devices, wherein one injector provides a first dose and second injector provides a second dose, both of which are delivered by manual injection. In still other embodiments, included in the kit or packaging system are two injectors as separate devices, wherein one injector provides a first dose administered by manual injection and the second injector provides a second dose administered by automatic injection. In yet other embodiments, included in the kit or packaging system are two injectors as separate devices, wherein one injector provides a first dose delivered by auto injection and the second injector provides a second dose delivered by manual injection.

In some embodiments, the kit or packaging system further comprises written instructions for administering the first and second injectable dosage forms comprising an epinephrine solution as described herein.

In certain embodiments, the written instructions provide that (a) the first injectable dosage form comprising an epinephrine solution as described herein is first administered to a patient to provide a therapeutic effect in response to an allergic emergency; and (b) subsequent to the first administration of the first injectable dosage form comprising an epinephrine solution, a second dose of an injectable dosage form comprising an epinephrine solution is administered to the patient.

In other embodiments, the kit or packaging system further comprise an article for holding and/or storing the first and second injectable dosage forms comprising an epinephrine solution as described herein. In one embodiment, the kit or packaging system comprises a carrying case. Examples of articles useful in the kits or packaging systems described herein can be found throughout the art and include, for example, those described in U.S. Pat. Nos. 4,044,933; 5,137,516; 6,405,912; 6,595,362; and 6,641,015 (each of which is incorporated by reference herein in their entirety); US Published Application No. 2005/0148933 (which is incorporated by reference herein in its entirety), and US Published Application No. 2004/0069667 (which is incorporated by reference herein in its entirety).

In certain other embodiments, the first and second injectable dosage forms can be contained within a protective liner or pouch. In one such embodiment, the protective liner or pouch can prevent damage due to moisture, light, or oxygen. In another such embodiment, the protective liner is a polymer-lined foil. Examples of protective liners or pouches useful in the kits or packaging systems described herein can be found throughout the art and include, for example, those described in US Published Application No. 2004/0182736 (which is incorporated by reference herein in its entirety), and U.S. Published Application No. 2003/0106824 2004 (which is incorporated by reference herein in its entirety).

In yet other embodiments, the injectable doses are identified in the kit or packaging system. In one embodiment, the injectable doses are identified by numerical markings or by location within the kit or packaging system. In another embodiment, the identification of the injectable doses indicates the order in which the doses are administered to the patient.

In other aspects of the present invention, provided herein are injectable dosage forms comprising the epinephrine solutions as described herein, wherein the injectable dosage forms further comprise a label comprising written instructions for administering the injectable dosage form. Examples of labels compatible with the injectable dosage forms described herein can be found throughout the art and include, for example, those described in U.S. Published Application No. 2007/0031619 (which is incorporated by reference herein in its entirety). In some embodiments, the injectable dosage form is an injector comprising a label that provides instructions for delivering a first dose and a second dose of an epinephrine solution by automatic injection from the same device. In other embodiments, the injectable dosage form is an injector comprising a label that provides instructions for delivering a first dose and a second dose of an epinephrine solution by manual injection from the same device. In still other embodiments, the injectable dosage form is an injector comprising a label that provides instructions for delivering a first dose by manual injection and the second dose by automatic injection. In yet still other embodiments, the injectable dosage form is an injector comprising a label that provides instructions for delivering a first dose manual injection and a second dose by automatic injection from the same device. In one embodiment of the injectable dosage form, the first dose of an epinephrine solution comprises less than about 0.15 mg of epinephrine. In another embodiment, the first dose of an epinephrine solution comprises at least about 0.5 mg of epinephrine.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### EXAMPLES

The following ingredients, processes and procedures for practicing the methods described herein correspond to that described above. The procedures below describe specific embodiments of methods of administering the injectable epinephrine dosage forms as described herein. Any methods or materials not particularly described in the following examples are within the scope of the invention and will be apparent to those skilled in the art with reference to the disclosure herein.

### Example 1

Administration of a first and second injectable dosage form comprising an epinephrine solution for the treatment of anaphylaxis

A patient weighing approximately 15 Kg experiencing an allergic emergency whereupon a caretaker initiates treatment at the onset of shortness of breath by administering to the patient an auto-injection of a first dose of an epinephrine solution comprising 0.1 mg of epinephrine intramuscularly. After approximately 5 minutes pass without amelioration of the symptoms of anaphylaxis, the caretaker manually administers to the patient a second dose of an epinephrine solution comprising 0.15 mg of epinephrine intramuscularly from the same device as the first injectable dose. Within about five minutes after the administration of the second injectable dose of epinephrine, the patient's symptoms of anaphylaxis are relieved or are at an acceptable level for transporting the patient to a physician for additional medical attention.

### Example 2

Administration of a first and second injectable dosage form comprising epinephrine for the treatment of anaphylaxis

A patient weighing approximately 25 Kg experiencing an allergic emergency initiates treatment at the onset of shortness of breath by self administering a manual injection of a first dose of an epinephrine solution comprising 0.125 mg of epinephrine intramuscularly. After approximately 5 minutes pass without amelioration of the symptoms of anaphylaxis, the patient manually self administers a second dose of an epinephrine solution comprising 0.125 mg of epinephrine intramuscularly from the same device as the first injectable dose. Within about five minutes after the administration of the second injectable dose of epinephrine, the patient's symptoms of anaphylaxis are relieved or are at an acceptable level for transporting the patient to a physician for additional medical attention.

### Example 3

Administration of a first and second injectable dosage form comprising epinephrine for the treatment of anaphylaxis

A patient weighing approximately 90 Kg experiencing an allergic emergency initiates treatment at the onset of shortness of breath by self administering an auto-injection of a first dose of an epinephrine solution comprising 0.6 mg of epinephrine subcutaneously. After approximately 5 minutes pass without amelioration of the symptoms of anaphylaxis, the patient manually self administers a second dose of an epinephrine solution comprising 0.75 mg of epinephrine intramuscularly from the same device as the first injectable dose. Within about five minutes after the administration of the second injectable dose of epinephrine, the patient's symptoms of anaphylaxis are relieved or are at an acceptable level for transporting the patient to a physician for additional medical attention.

### Example 4

Administration of a first and second injectable dosage form comprising epinephrine for the treatment of anaphylaxis

A patient weighing approximately 110 Kg experiencing an allergic emergency initiates treatment at the onset of shortness of breath by self administering an auto-injection of a first dose of an epinephrine solution comprising 0.75 mg of epinephrine intramuscularly. After approximately 5 minutes pass without amelioration of the symptoms of anaphylaxis, the patient self administers a second dose of an epinephrine solution from an auto-injector comprising 1.0 mg of epinephrine intramuscularly from the same device as the first injectable dose. Within about five minutes after the administration of the second injectable dose of epinephrine, the patient's symptoms of anaphylaxis are relieved or are at an acceptable level for transporting the patient to a physician for additional medical attention.

### Example 5

A kit comprising a single device containing a first and second dose of an epinephrine solution in two injectable dosage forms for the treatment of anaphylaxis

A kit is provided which contains a first and second dose of an epinephrine solution in injectable dosage forms wherein the epinephrine solution in both the first and second dose comprises 0.1 mg of epinephrine. The two injectable dosage forms are for auto-injection and are administered from a single device, which is sealed in a foil-lined plastic pouch.

The kit further contains written instructions to aid the patient in administering the injectable dosage forms of epinephrine contained therein in the correct order and at the correct time.

The instructions provide as follows: (a) the first injectable dosage form, labeled as 1, is to be administered intramuscularly by the patient; (b) if the symptoms of anaphylaxis do not improve or terminate within approximately ten minutes, the second injectable dosage form, labeled as 2, is to be administered intramuscularly.

The written instructions further provide standard information including the proper storage conditions for the injectable dosage forms, how to properly dispose of the unused dosage forms, contra-indications related to dosage forms comprising epinephrine, etc.

A carrying case is also included in the kit which provides easy storage for the injectable dosage forms and also provides additional protection from moisture, light and oxygen.

### Example 6

A kit comprising a first and second dose of an epinephrine solution in two injectable dosage forms for the treatment of anaphylaxis

A kit is provided which contains a first and second dose of an epinephrine solution in injectable dosage forms wherein the epinephrine solution in the first injectable dosage form comprises 0.5 mg epinephrine and the epinephrine solution in the second injectable dosage form comprises 0.75 mg of epinephrine. The two injectable dosage forms are for manual injection and are contained is separate auto-injecting devices, each of which is sealed in a foil-lined plastic pouch.

The kit further contains written instructions to aid the patient in administering the injectable dosage forms of epinephrine contained therein in the correct order and at the correct time.

The instructions provide as follows: (a) the first injectable dosage form comprising 0.5 mg epinephrine, labeled as 1, is to be administered intramuscularly by the patient; (b) if the symptoms of anaphylaxis do not improve or terminate within approximately fifteen minutes, the second injectable dosage form comprising 0.75 mg epinephrine, labeled as 2, is to be administered intramuscularly.

The written instructions further provide standard information including the proper storage conditions for the injectable dosage forms, how to properly dispose of the unused dosage forms, contra-indications related to dosage forms comprising epinephrine, etc.

A carrying case is also included in the kit which provides easy storage for the injectable dosage forms and also provides additional protection from moisture, light and oxygen.

We claim:

1. A method of treating an allergic emergency in a patient, comprising injecting into a patient in need thereof a first dose of an epinephrine solution comprising less than about 0.15 mg of epinephrine and optionally subsequently injecting into the patient a second dose of an epinephrine solution, wherein the first and second doses are administered from a single device.

2. The method of claim 1, wherein the amount of epinephrine in the first dose is about the same as the amount of epinephrine in the second dose.

3. The method of claim 2, wherein the amount of epinephrine in the first dose is about 0.1 mg.

4. The method of claim 2, wherein the amount of epinephrine in the first dose is about 0.05 mg.

5. The method of claim 1, wherein the amount of epinephrine in the first dose is greater than the amount of epinephrine in the second dose.

6. The method of claim 5, wherein the amount of epinephrine in the second dose is about 0.1 mg.

7. The method of claim 5, wherein the amount of epinephrine in the second dose is about 0.05 mg.

8. The method of claim 1, wherein the amount of epinephrine in the first dose is less than the amount of epinephrine in the second dose.

9. The method of claim 8, wherein the amount of epinephrine in the second dose is at least about 0.15 mg.

10. The method of claim 8, wherein the amount of epinephrine in the second dose is at least about 0.3 mg.

11. The method of claim 1, wherein the first dose is automatically injected.

12. The method of claim 11, wherein the second dose is automatically injected.

13. The method of claim 11, wherein the second dose is manually injected.

14. The method of claim 1, wherein the first dose is manually injected.

15. The method of claim 14, wherein the second dose is automatically injected.

16. The method of claim 14, wherein the second dose is manually injected.

17. The method of claim 1, wherein the second dose is injected less than about 30 minutes after the first dose.

18. The method of claim 17, wherein the second dose is injected less than about 20 minutes after the first dose.

19. The method of claim 17, wherein the second dose is injected less than about 10 minutes after the first dose

20. The method of claim 17, wherein the second dose is injected between about 2 to about 10 minutes after the first dose.

21. The method of claim 1, wherein the patient weighs between about 5 to about 15 Kg.

22. The method of claim 1, wherein the concentration of the epinephrine solution in the first dose is about 1 mg/ml.

23. The method of claim 1, wherein the concentration of the epinephrine solution in the first dose is about 0.5 mg/ml.

24. A method of treating allergic emergency in a patient, comprising injecting into a patient in need thereof a first dose of an epinephrine solution comprising at least about 0.5 mg of epinephrine and optionally subsequently injecting into the patient a second dose of an epinephrine solution, wherein the first and second doses are administered from a single device.

25. The method of claim 24, wherein the amount of epinephrine in the first dose is about the same as the amount of epinephrine in the second dose.

26. The method of claim 25, wherein the amount of epinephrine in the first dose is about 0.5 mg.

27. The method of claim 25, wherein the amount of epinephrine in the first dose is between about 0.5 mg to about 1.0 mg.

28. The method of claim 25, wherein the amount of epinephrine in the first dose is about 0.75 mg.

29. The method of claim 24, wherein the amount of epinephrine in the first dose is greater than the amount of epinephrine in the second dose.

30. The method of claim 29, wherein the amount of epinephrine in the second dose is about 0.5 mg.

31. The method of claim 29, wherein the amount of epinephrine in the second dose is about 0.3 mg.

32. The method of claim 29, wherein the amount of epinephrine in the second dose is about 0.15 mg.

33. The method of claim 29, wherein the amount of epinephrine in the second dose is less than about 0.15 mg.

34. The method of claim 24, wherein the amount of epinephrine in the first dose is less than the amount of epinephrine in the second dose.

35. The method of claim 24, wherein the first dose is automatically injected.

36. The method of claim 35, wherein the second dose is automatically injected.

37. The method of claim 35, wherein the second dose is manually injected.

38. The method of claim 24, wherein the first dose is manually injected.

39. The method of claim 38, wherein the second dose is automatically injected.

40. The method of claim 38, wherein the second dose is manually injected.

41. The method of claim 24, wherein the second dose is injected less than about 30 minutes after the first dose.

42. The method of claim 41, wherein the second dose is injected less than about 20 minutes after the first dose.

43. The method of claim 41, wherein the second dose is injected less than about 10 minutes after the first dose.

44. The method of claim 41, wherein the second dose is injected within about 2 to about 10 minutes after the first dose.

45. The method of claim 24, wherein the patient weighs between about 50 Kg to about 100 Kg.

46. The method of claim 24, wherein the concentration of the first dose of epinephrine solution is 1 mg/ml.

47. The method of claim 24, wherein the concentration of the first dose of epinephrine solution is 0.5 mg/ml.

48. A kit or packaging system for treating an allergic emergency, comprising a first injectable dosage form comprising an epinephrine solution comprising less than about 0.15 mg of epinephrine and a second injectable dosage form comprising an epinephrine solution.

49. The kit or packaging system according to claim 48, further comprising an article for holding said first and second injectable dosage forms and written instructions for administering said first and second injectable dosage forms.

50. The kit or packaging system according to claim 48, wherein said first and second injectable dosage forms are administered from a single device.

51. A kit or packaging system for treating an allergic emergency, comprising a first injectable dosage form comprising an epinephrine solution comprising at least about 0.5 mg of epinephrine and a second injectable dosage form comprising an epinephrine solution.

52. The kit or packaging system according to claim 51, further comprising an article for holding said first and second injectable dosage forms and written instructions for administering said first and second injectable dosage forms.

53. The kit or packaging system according to claim 51, wherein said first and second injectable dosage forms are administered from a single device.

54. A drug delivery device containing an epinephrine solution, wherein the device is capable of delivering a first dose of an epinephrine solution comprising less than about 0.15 mg of epinephrine and optionally a second dose of an epinephrine solution.

55. A drug delivery device containing an epinephrine solution, wherein the device is capable of delivering a first dose of an epinephrine solution comprising about 0.5 mg of the epinephrine and optionally second dose of an epinephrine solution.
